(19) United States
(12) Patent Application Publication
(10) Pub. No.: US 2011/0214670 A1
(43) Pub. Date: Sep. 8, 2011

(54) Bite block

(76) Inventor: Louis G. Kost, Owensboro, KY (US)

(21) Appl. No.: 12/660,695

(22) Filed: Mar. 2, 2010

(51) Int. Cl. A61M 16/00

(52) U.S. Cl. 128/200.26

(57) ABSTRACT

A bite block that includes first and second members interconnected by screw threads on the respective members. The first member is a relatively rigid body having an open ended hole with internal screw threads on at least a portion of the length thereof. The second member is an elongate open ended sleeve having screw threads on the outer surface thereof that thread into the hole in the body member. A slit in the wall of the sleeve accommodates a reduction in the internal diameter of the sleeve. A selected one of the internal and external threads progressively decreases in diameter in a direction so as to cause the sleeve internal diameter to decrease as the sleeve is threaded into the body member.

### RELATED APPLICATIONS

This application and claims priority from U.S. application Ser. No. 12/384,247 filed on Apr. 2, 2009 hereby incorporated by reference in its entirety.

### FIELD OF INVENTION

This invention relates to a mechanism, known as a bite block, to prevent a person from biting through an oral and/or tracheal airway tube inserted into the mouth of a patient.

### BACKGROUND OF INVENTION

Prior known devices used to perform this task use an anchoring method like a hook, loop, strap, tape or other means to keep the bite block locked in place around the airway. Some known devices have create sharp edges, pressure and additional spiky jagged ends that can cause cuts, skin abrasions and pressure sores on the patients surrounding skin. By way of examples of known bite blocks reference maybe had to the following U.S. Pat. No. 5,421,327 issued on Jun. 6, 1995 naming inventors Mary E Flynn et al and entitled “Bite Block Having Finger-Accommodating Openings”.

### SUMMARY OF INVENTION

The bite block of the present invention addresses deficiencies of previous bite blocks and is characterized principally by the provision permitting the bite block to grasp the airway tube (endrotracheal tube, or any other cylindrical airway) without using the prior mentioned methods. More particularly there is provided in accordance with the present invention a bite block A bite block for use with a patient airway tube having a preselected outer diameter, said bite block comprising; consisting essentially of or consisting of a first relatively rigid body member, an open ended hole of selected length extending through the body member and internal screw threads on at least a portion of the length of the hole. A second member comprises, consist essentially of or consists of an elongate open ended sleeve having a preselected wall thickness and in a normal at rest state a preselected internal diameter, a slit in the wall that extends from one end thereof in a direction toward the opposite end, and external screw threads on the sleeve extending from the one end in a direction toward the opposite end. The internal and external threads being inter-engagable in mating relation detachably interconnecting the sleeve and the body member. A selected one of the internal and external threads progressively decrease in diameter so that with the bite block in an assembled state the sleeve internal diameter gradually decreases to a selected minimum diameter smaller than the outer diameter of a tube associated therewith as the sleeve is threaded into the body member.

By having the bite block tightly grasp the airway tube it is independent and secure with the need for a separate anchoring means that can hinder and obstruct access to the airway.

The bite block is preferably made of a transparent (clear) plastic, such as a silicon material, plastisol, elastomer, or other thermoplastic, rubber, or combinations thereof material and this allows the health care practitioner to visually see the airway features and centimeter markings on the oral or tracheal tube making it easier to note the correct position of the tube.

Other objects, features, and advantages of the invention will be apparent with the following detailed description taken in conjunction with the accompanying drawings showing a preferred embodiment of the invention.

### BRIEF DESCRIPTION OF THE DRAWINGS

A better understanding of the present invention will be had upon reference to the following description in conjunction with the accompanying drawings in which like numerals refer to like parts throughout the several views and wherein:

FIG. 1 is a view illustrating applicants bite block mounted on an airway tube;

FIG. 2 is an exploded, part sectional, view of the bite block shown in FIG. 1;

FIG. 3 is a is a sectional view taken essentially along line 3-3 of FIG. 2; and

FIG. 4, similar to a portion of FIG. 2, illustrates modifications to a portion of the bite block.

### DETAILED DESCRIPTION OF THE PREFERRED EMBODIMENT

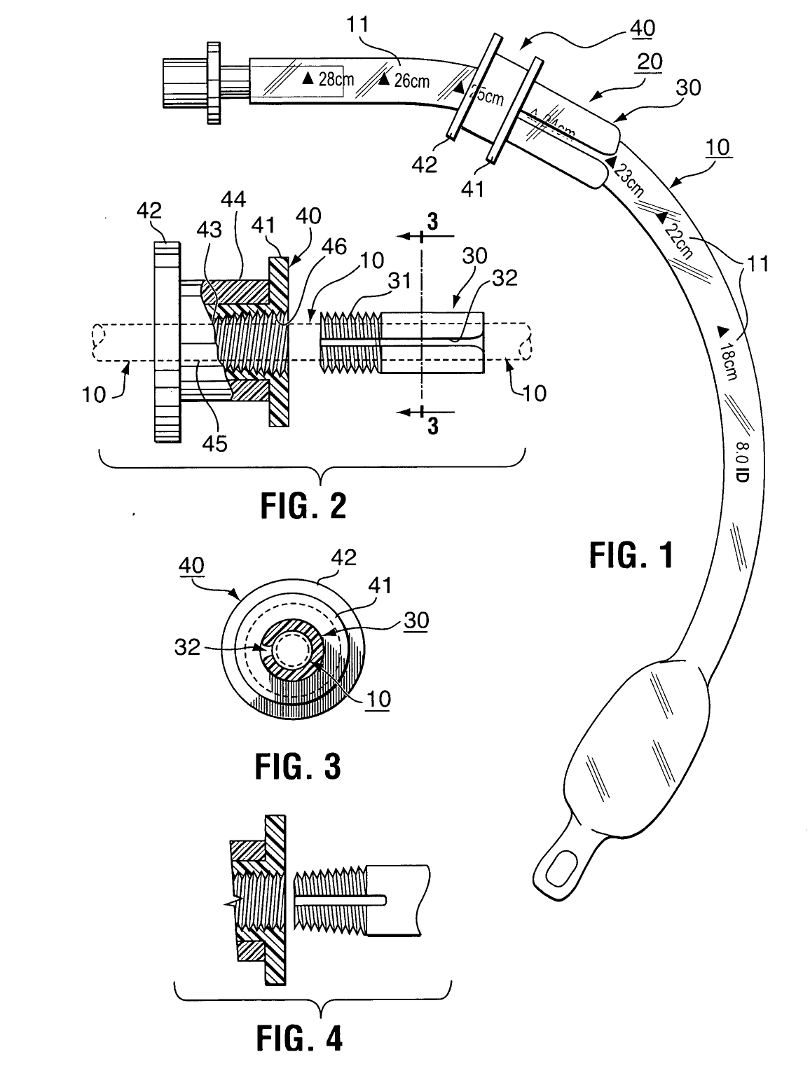

Illustrated in the drawing FIGS. 1-4 is a known airway tube 10 having applicants bite block 20 mounted thereon. The bite block has a through hole that allows the tube to pass there-through. Airway tubes are available with differing outer diameters dictated generally by whether the patient is an adult or child, male or female. At least a portion of the through hole is selectively adjustably variable in diameter from a maximum diameter, that is slightly greater than the outer diameter of a tube to used in association therewith, to a minimum diameter that is smaller than the outer diameter of such tube. The portion that can be reduced in diameter is resiliently biased to it's maximum diameter which is its normal at rest state.

Referring now in more detail to the drawing the bite block 20 comprises respective first and second components 30, 40 in which the first component 30 is an elongate open ended tubular sleeve having an externally threaded portion 31 extending from one end of the sleeve in a direction toward the other end. There is a slit 32 in the wall of the sleeve that extends from the above mentioned one end in a direction toward the other end. In the preferred embodiment, illustrated in FIG. 2, the slit 32 extends along the entire length of the tubular sleeve while in the embodiment illustrated in FIG. 4 the slit extends only along a portion of the length of the sleeve. The purpose of the slit is to accommodate changing the internal diameter of the sleeve from it's normal at rest state of maximum diameter that is greater than the outer diameter of the tube 10 being used in association therewith to a minimum diameter that is smaller than that outer diameter of the tube 10. At this minimum diameter the bite block tightly grips the tube 10.

The sleeve 30 is preferably made of a transparent plastics material permitting progressive tube length designations or indicia 11 imprinted, printed, or formed into the tube 10 to be seen through the wall of the sleeve. The wall thickness of the sleeve is chosen taking into account the stiffness of the material such that the sleeve internal diameter can be readily varied using only finger grip to rotate the components relative to one another about the longitudinal axis of the sleeve 30. The characteristics of the material such as resilience, flexibility and elasticity are considered and chosen so that the sleeve is resiliently biased to a normal at rest maximum diameter and permitting repeated usage by staying within the elastic limits as the sleeve is deformed.

The second component 40 is a rigid or relatively stiff body member made of a plastics, metal, or rubber like material or combinations thereof. The body has spaced apart ridges 41, 42 interconnected by a hub 43. Ridge 41 projects further from the hub than the ridge 42 and when in use abuts against the outer upper and lower lip portions of the patient. The hub 43 has a layer 44 of soft resilient material located, at least, in appropriate positions for abutting the patients respective upper and lower teeth. This padding of softer material may, for example, be a suitable plastics material such as neoprene or a rubber or rubber like material that protects the teeth from chipping or other damage. The ridges 41, 42 maybe circular flanges with one having a larger diameter than the other as shown and together with the hub interconnecting the same provide a body having the general appearance of a spool. The hub of the spool is surrounded by an annular cushioning member. Alternatively the flanges 41 and 42, at minimum, could each consist of a pair of ribs projecting radially in opposite directions away from the hub.

There is an open ended hole 45 through the body member 40 with at least a portion thereof having internal screw threads 46. In the embodiment illustrated in FIG. 2 these threads are located in a tapered portion decreasing in diameter in a direction from flange 41 toward flange 42. The sleeve 30 external threads 31 mate with the threads 46 and as components 30, 40 are rotated relative to one another to increase penetration of component 30 into component 40 the tapering of the threads causes the internal diameter of the sleeve to progressively decrease in size. The width of the slit 32 is so chosen as to allow a selected reduction in diameter. The amount of taper of the threads is chosen such that the reduction in diameter is sufficient for the sleeve to tightly grasp the tube 10 used in association therewith and without exceeding the elastic limit of the sleeve.

To use the bite block the sleeve is slide over the airway tube just past the patients teeth after such tube has placed in the trachea. While holding the sleeve the body portion of the bite block is slid onto the tube and rotated to thread it onto the sleeve. Rotation of the body portion is stopped when the bite block is secure on the tube and the patients teeth are positioned on the soft outer hub portion of the body member.

The foregoing detailed description is given primarily for clearness of understanding and no unnecessary limitations are to be understood therefrom, for modification will become obvious to those skilled in the art upon reading this disclosure and may be made upon departing from the spirit of the invention and scope of the appended claims. Accordingly, this invention is not intended to be limited by the specific exemplifications presented herein above. Rather, what is intended to be covered is within the spirit and scope of the appended claims.

1. A bite block for use with a patient airway tube having a preselected outer diameter, said bite block comprising;
(A) a first relatively rigid body member, an open ended hole of selected length extending through said body member, internal screw threads on at least a portion of the length of said hole; and
(B) a second member comprising an elongate open ended sleeve having a preselected wall thickness and in a normal at rest state a preselected internal diameter, a slit in said wall that extends from one end thereof in a direction toward the opposite end, external screw threads on said sleeve extending from said one end in a direction toward said opposite end, said internal and external threads being inter-engagable in mating relation detachably interconnecting said sleeve and said body member, a selected one of said internal and external threads progressively decreasing in diameter so that with the bite block in an assembled state the sleeve internal diameter gradually decreases to a selected minimum diameter smaller than the outer diameter of a tube associated therewith as the sleeve is threaded into the body member.

2. The bite block as defined in claim 1 wherein said slit extends at least throughout the length of said external threads on said sleeve.

3. The bite block as defined in claim 1 wherein said slit extends throughout the entire length of said sleeve.

4. The bite block as defined in claim 1 wherein at least said sleeve is made of a transparent plastics material.

5. The bite block as defined in claim 1 wherein the internal threads in said body member progressively decrease in diameter.

6. The bite block as defined in claim 1 wherein said body member comprises a hub, a first rib means on one end thereof and a second rib means on the opposite end, said first and second rib means being spaced apart a selected distance from one another and projecting outwardly in a direction radially from said hub with said first rib means projecting a further distance than said second rib means.

7. The bite block as defined in claim 6 wherein said first rib means comprises a first annular flange.

8. The bite block as defined in claim 7 wherein said second rib means comprises a second annular flange.

9. The bite block as defined in claim 8 wherein said first and second annular flanges are circular in outline shape about an axis parallel to the axis of the hole through said body member.

10. The bite block as defined in claim 6 wherein said hub has a resiliently outer surface.

11. The bite block as defined in claim 1 wherein said body member comprises a cylindrical hub, a first flange and a second flange disposed respectively at respective opposite ends of said hub, said flanges being spaced apart a selected distance from one another and projecting in a direction radially from the hub with said first flange projecting further than said second flange and a resilient outer surface on said hub.

12. The bite block as defined in claim 11 wherein said soft outer surface surrounds said hub.
